(19) United States
(12) Patent Application Publication
(10) Pub. No.: US 2022/0362400 A1
(43) Pub. Date: Nov. 17, 2022

(54) PIVOTING ELECTRODYNAMIC COMPOSITION AND MEDICAMENT

(71) Applicant: Exothule Corporation, Irvine, CA (US)

(72) Inventor: Peter Butzloff, Walnut, CA (US)

(21) Appl. No.: 17/874,669

(22) Filed: Jul. 27, 2022

Related U.S. Application Data
(63) Continuation-in-part of application No. PCT/US20/32976, filed on May 14, 2020.
(60) Provisional application No. 62/977,352, filed on Feb. 16, 2020.

(51) Int. Cl. A61K 47/69; A61K 47/54

(52) U.S. Cl. CPC A61K 47/6949; A61K 47/549

(57) ABSTRACT

A first derivative of a fullerene is covalently bonded to an adenosine phosphate such as adenosine triphosphate, adenosine diphosphate, adenosine monophosphate, or cyclic adenosine monophosphate. A second fullerene is covalently bonded to a second type of functional group including amines of arginine and lysine. The second fullerene is then van-der-Waals bonded to the first fullerene, to form a biaxially pivoting fullerene molecular composition. This composition can be treated to intercalate and carry a drug or an antibody for later release by directed irradiation. The injected composition with optional drug carrier is electrodynamically activated by irradiation of the injected target organ or tissues by the application of, for example, radio frequency (RF) energy to release the drug and lyse the targeted cells.

### CROSS-REFERENCE TO RELATED APPLICATIONS

This application is a Continuation-in-Part of International Application PCT/US20/32976 filed on May 14, 2020 which claims the benefit of U.S. provisional patent application No. 62/977,352 filed on Feb. 16, 2020 both entitled “Pivoting Electrodynamic Composition and Medicament,” and both incorporated herein in their entireties.

### BACKGROUND OF THE INVENTION

### Field of the Invention

The invention is related to electrodynamic fullerene compositions and pivoting biaxial electrodynamic fullerene compositions (EPF).

### Description of the Prior Art

Human cells interact quite differently with commensal microbes according to their interdependent and sometimes pathogenic properties as these microbes compete, while surviving inside the human body. The effects of surface charges in contact with water, proteins, and lipid membranes become differently engaged in these interactions.

The study of fungi is generally termed mycology. Fungi are classified to be in a different kingdom from plants, bacteria, or animals. Like animals, fungi secrete enzymes to break down biopolymers into simple sugars and are well known to decompose both living and dead organisms to obtain energy and grow. Fungi are distinguished by the presence of chitin, a complex sugar biopolymer that is present in their cell walls. When a virus colonizes a fungus as a host, this is known as a mycovirus or mycophage. It is commonly understood that virus particles usually colonize bacteria which are present as hosts; in these cases, the invading virus is termed a bacteriophage. The simultaneous predation on both fungi and bacteria hosting of one type of phage (virus) characterizes these virus particles as a mycobacteriophage.

Virus particles are by far the largest mass of evolving carbon-based constructs on planet Earth, wherein most of those viruses colonize or live within bacteria. About 99.99% of viruses are benign, live in balance with their hosts, and do not cause immediate damage to the cells of animals or human beings. The healthy human being is a hierarchy of commensal organisms, where most of the living tissues of a person are microbial, and about 10% of the total genetic mass belongs to cells we recognize to be human; this collection of creatures we call ourselves is therefore better holistically described as a holobiont. Indeed, the loss of some of our symbionts can cause some types of disease. Therefore, any composition that attempts to correct for a disease, had better not create a dysbiosis, or disruption of beneficial microbial relationships that humans rely on to live and help digest food in the process of extracting nutrients.

The highest specialization of animals is neural tissue, where neurons have the greatest need for energy, and therefore also obtain the highest concentration of energy harvesting mitochondria in their structures. In all life, polyphenols have evolved as internal cellular control molecules to regulate the cellular biology of bacteria, plants and animals. Plants have evolved the use of polyphenols as a primary defense against fungal and bacterial invasion. The human consumption of plant derived flavonoids, phytoestrogens, and non-flavonoid polyphenols confer a wide range of long-term nutritional and health benefits. Polyphenols modulate cellular signaling pathways by interacting with molecular receptors to control tissue dilation, inflammation, and to affect the proper function of neurons. Polyphenols interact with neurotransmitters to have a direct effect on cognitive and cerebrovascular, or brain blood flow functions. Mitochondria function as energy harvesting organelles, or sites inside cells where glucose can be used to build proteins and peptides used to build the cellular structure. At least one mitochondrion is present at every branch point of every dendrite in a neuron, and one mitochondrion is always at the growth tip of each dendrite, called the filopodia.

A delicate chemical balance of reduction and oxidation (REDOX) operates mitochondria and drives cellular function, especially neural function, which is the most energy intensive and therefore the most reliant on mitochondria for energy. Nowhere is microbial infection more damaging than in neural tissues, and especially so in the brain. Neural mitochondria can become compromised when virus particles attempt to parasitize the nuclear and mitochondrial genetic processes. The brains of higher organisms have therefore evolved significant redundancy to address viral infection, by becoming larger and more complex. Therefore, it is generally accepted that viruses having host-microbe interactions within animal tissues are responsible for all large brain structure expansion, expressed evolutionarily within all animal life on earth. Because virus particles sometimes recombine and alter their genetic structures to change over time, it is quite likely that the human brain has evolved sporadically to address and adapt to recurrent neural viral infections, by building increased redundancy.

Even as internal isolation barriers, such as the blood brain barrier, aim to prevent most microbial infections from destroying the brain and neural tissues, dietary polyphenols as cell signal molecules are often ineffective in either their recruitment capacity or their antimicrobial capacity to address the viral load in cholesterol or lipid rich regions of cells, being that they prefer to solvate in water rich regions and tend to avoid lipid or cholesterol phase cell membranes. The most sensitive region of the cell where a virus may hide, is therefore in the cholesterol containing membrane that encapsulates the mitochondria, called the endoplasmic reticulum. This is the membrane through which glucose must pass, to change adenosine mono phosphate (AMP), or adenosine diphosphate (ADP) to adenosine triphosphate (ATP), by redox chemistry with active oxygen species, necessary to generate chemical energy and build proteins for use in cellular biomolecules. Animal cells have therefore used the REDOX chemistry within mitochondria to deliberately generate reactive oxygen species (ROS) both to detoxify invasive proteins, and to defend the cell against invasive virus particles having a protein coating around each virus particle, called viral capsids. Of particular interest are the membrane budding viruses, such as the various types of influenza, and herpes simplex virus (HSV), Ebola virus, and also another virus type that has proven to be quite good at hiding from the immune system within phospholipid cell membranes, the human immune deficiency virus (HIV). These viral types are intimately involved with or are greatly amplified in virulence by the acquisition of Adenosine Triphosphate (ATP) and the presence of glucose sugars in the electron charge transfer cycle of cellular respiration.

The immune response of higher animals has evolved epigenetic marks such as DNA methylation, histone modification, and changes in populations of microRNA that are involved in the transfer of immunity and nutritional history to impact heritable immune responses and metabolic modifications so that real experiences in the environment transfer to the phenotype of expressed traits transcending multiple generations. However, the confluence of the immune system training with mitochondrial ROS has drawbacks. Over time, the individual immune response becomes less able to address environmental assaults, so that the production of defensive ROS must become greater in the ageing organism. This means greater ROS to deflect invasive virus particles and other microbes, also results in increased cellular genetic damage by self-oxidation. Overproduction of ROS then leads to cell senescence and cellular self-termination at the end of a certain number of cell divisions. The observation of an upper average limit on cell division was first discovered and reported by Hayflick in 1969 and is generally known today as the Hayflick limit. The Hayflick limit is now known to be controlled by mitochondria in each species, where the lifespan of that organism is at least partly determined by the amount of ROS generated by the mitochondria in that species. The release of ROS by mitochondria is characterized by hydrogen peroxide (H₂O₂), and a wide variety of biological molecules involved with REDOX control, including Thioredoxin Interacting Protein or TXNIP, and telomerase, which is involved with control of mitochondrial defects resulting from extensive ROS damage. The shortening of telomeres arising from excessive ROS generation eventually exposes DNA to oxidative damage and increases the rate of cell senescence. The presence of long-term latent virus particles, such as herpes simplex and cytomegalovirus hiding in cell membranes, are a chronic cause of ROS generation and telomere shortening, and the confluence of these are implicated in all long-term neural dysfunction and human mental illness.

The herpes viruses, called herpetic virus, of the Herpesviridae family, infects most people worldwide in both developing and developed countries. Cytomegalovirus (CMV) is the herpes virus, of which herpes simplex 1 is associated with the common cold sore. Because about 90% of the world population is infected with herpetic virus, it is likely that everyone is eventually exposed at least once and probably many times during their lifetimes. Following initial infection, CMV establishes a lifelong latent infection, with likely reactivation and reinfection at later dates. In particular, most investigations have shown an association of herpes simplex virus type 1 (HSV-1) with Alzheimer's disease (dementia), but it was not until recently that that a complex chain of events relating to the role of ROS in the etiology of this disease could be clarified. Many reports now suggest that the long-term effects of what apparently begins as a mild short-term viral infection such as influenza or cold sores, could continue to impair the cognitive functions of infected subjects long after the original symptoms abate. Latent phase infection effects may contribute to a wide range of mental illness, impairments, and accumulated cognitive decline, especially cardiovascular disease, and bipolar disorder. It is of special and major concern, however, that the total impact of hepatic viruses and CMV on mental health and human intelligence, may be quite high.

It has been determined that the Herpes Simplex Virus (HSV) and influenza virus require the use of mitochondrial energy compound adenosine triphosphate (ATP) to replicate, and that inhibition or depletion of cellular ATP blocks the maturation of the viral sheath proteins, especially the viral protein 26 or VP26 that is used to form the reproduced HSV virus. While this is interesting, it is also notable that depletion of ATP inevitably leads to cell death from lack of energy to perform respiration and build essential cellular proteins. Mitochondria exhibit a condensed structure of the cristae, indicating the characteristic state of active respiration. More subtle analysis has led researchers to conclude that the VP26 of HSV-1 requires ATP to form the correct angles of the capsid plates to sheath the virus in its protein case during this process. Unfortunately, aside from vaccines that target antibodies to the outside protein coating or capsid of virus particles, no more generally effective strategy has yet been invented or formulated to take medical advantage of well understood ATP recruitment by budding viruses.

One method to improve the human physical condition and cognitive well-being to combat viruses budding from lipid membranes can be achieved by a careful design consideration of cholesteric affinity to enable a synergy with the evolutionary defense at cellular membranes. Such molecules should also operate to confer protective functions to the normal operation of mitochondria, especially those mitochondria in human neural cells and brain tissue. Zanamivir® and Oseltamivir® are antiviral drug examples of molecules having both a lipophilic end to interface with hydrophobic cellular membranes, and ionic portions, usually containing an amine group that allow these ends to interface will with the cellular cytosol. Yet these tools are limited in their ability to help control influenza pandemics or confer immunity to chronic viral infections. These industrial examples are only one part of a complex biological solution, genetic factors, environmental immunity reinforcement, and physical training play interactive roles in the extension of healthy cellular homeostasis.

There is considerable basic science and epidemiological evidence that infectious agents may be contributing to the neuropathology and clinical manifestations of Alzheimer's disease and other neuropathology, and that the herpes simplex virus particle types are present and implicated in these diseases. This viral hypothesis has been in the literature for many decades, and evidence to support this hypothesis has been widely supported. HSV and other viruses are usually also infectious to bacteria, which provide an interesting way to hide from the human immune system to induce recurrent infection via the many microbes that are commensal to the gut and beneficial or used for the survival of humans. Existing treatments for chronic viral initiated diseases have failed, and the need for extended care incurs severe economic costs as well as impairment of the quality of human life in aged individuals. The failure of treatments tested in clinical trials in patients with Alzheimer's Disease during the last decades, together with demographic increases in the age of our populations, underlies the urgency for new sorts of thinking to address these matters.

What is therefore needed is a multiplexed solution for effectively extinguishing influenza, as well as eradicating other budding or latent virus particles such as CMV. Desirably, a general treatment for budding viruses should include a prophylactic prevention of genetic or proteomic damage to human cells, especially neural cells, in response to infection.

### SUMMARY

These and other advantages of the present invention will be further understood and appreciated by those skilled in the art by reference to the following written specifications, claims and appended drawings.

The present invention provides a composition having a fullerene covalently bonded to a phosphate of adenosine where the phosphorus is in a +5 oxidation state. The fullerene is one of C60 fullerene or C70 fullerene, for example. Where “fullerene” is mentioned, it is to be understood to refer to any fullerene, including C60 fullerene and C70 fullerene. In a first fullerene, the fullerene is covalently bonded to one functional group of adenosine triphosphate (ATP), adenosine diphosphate (ADP), adenosine monophosphate (AMP), or cyclic adenosine monophosphate (cAMP). The fullerene derivative is additionally covalently bonded to a second functional group of ATP, ADP, AMP, or cAMP. A second fullerene is van-der-Waals bonded to the first fullerene, where the second fullerene is covalently bonded to at least one preselected amino acid and where the composition includes an electrodynamic biaxially pivoting fullerene pivot molecules. The second fullerene is covalently bonded to a first amino acid that includes arginine. Additionally, the second fullerene is covalently bonded to a first amino acid that includes lysine. Further, the second fullerene is covalently bonded to two amino acids that include arginine and lysine.

The invention also includes a method of exciting fullerene pivot molecules dispersed in an organ or region of treatment, where the treatment may be applied to a cancer, an organ, a portion of a limb, or the entire body of a person.

In one aspect, the method of excitation of fullerene molecular pivots is by the directed application of electromagnetic waves capable of penetrating tissues such as radio waves of greater than 7 GHz, or in some embodiments, electromagnetic infrared light waves being of greater than 800 nanometers.

In another aspect, the method of excitation of fullerene molecular pivots is by the directed application of electric energy pulses, where in some embodiments, the electric waves are sinusoidal or alternating current, and in other embodiments, the electric waves are square waves or can have triangular wave forms.

The invention also includes a method of preparing the foregoing composition, including adding an excess of dry crystalline powder of adenosine triphosphate (ATP) to solvent-free and dry crystalline powder of fullerene in a first predetermined ratio; combining a mixture of the dry crystalline powders in a shear grinding mill under shear pressure below 55° C. for about 15 minutes to produce fullerene-ATP; dissolving fullerene ATP into water and at least 10% glycerol solvent to make a dispersion, wherein the solvents are selected to expedite the delivery of medicament in the finished product mixture and wherein a fullerene-ATP dispersion is produced; adding an excess of dry crystalline powder of at least a first amino acid functional group (R1 ) and a second amino acid functional group (R2) to solvent-free and dry crystalline fullerene in a second predetermined ratio; combining a mixture of the dry crystalline (fullerene and R1 and R2 ) powders in a shear grinding mill under shear pressure below 55° C. for about 15 minutes to produce fullerene-R1-R2; dissolving fullerene-R1-R2 into water and at least 10% glycerol solvent to make a dispersion, wherein the solvents are selected to expedite the delivery of medicament in the finished product mixture and wherein a fullerene-R1-R2 dispersion is produced; combining the fullerene -ATP dispersion with the fullerene-R1-R2 dispersion in a mixer equipped with ultrasonic irradiation to produce an electrodynamic biaxial fullerene pivot; and actuating the electrodynamic biaxial fullerene pivot by irradiating it with ultrasound at about 200 watts and about 40 kilohertz for about 20 min. In one embodiment, R1 includes a first amine and R2 includes a second amine. In further embodiments, R1 includes L-Arginine (Arg) and R2 includes L-Lysine (Lys).

The present invention also includes a method of stimulating the foregoing composition by activating the composition by one of RF radiation or an applied electric current. The applied electric current can be either an AC current or a pulsed DC current, for example, and either at a frequency between about 5.0 GHz to about 11.5 GHz. The RF radiation can also be applied at a frequency between about 5.0 GHz to about 11.5 GHz. However, an upper limit of 10.6 GHz is recommended to avoid heating water in the affected tissues. In still further embodiments stimulating the composition comprises applying infra-red electromagnetic radiation with a wavelength from 800 nm to 1000 nm where living tissues are translucent.

The present invention includes yet another method of preparing the foregoing composition, including combining a dry crystalline powder of adenosine phosphate with solvent-free fullerene powder to produce a first mixture; shearing the first mixture in a shearing mill while maintaining a temperature below 55° C. to produce a covalently reacted first mixture; combining a dry crystalline amino acid powder with solvent-free fullerene powder to produce a second mixture; shearing the second mixture in a shearing mill while maintaining temperature below 55° C. to produce a covalently reacted second mixture; combining the first mixture with the second mixture to create a heterogeneous combination of each; adding about 20 parts water to one part of the combined mixture; irradiating the aqueous mixture for about 3 minutes with microwave radiation at 500 watts per liter to homogenize unlike fullerene derivatives by inductive van-der-Waals intercalation to produce homogenized ATP-fullerene pivot amino acid-fullerene conjugates; mixing ATP-fullerene pivot amino acid-fullerene into water containing about 10% glycerol to produce an ATP-fullerene pivot amino acid-fullerene conjugate solution; and mixing the ATP-fullerene pivot amino acid-fullerene solution with a predetermined substrate to form an ATP-fullerene pivot amino acid-fullerene formulation.

The present invention provides yet another method of preparing the foregoing composition, including combining a dry crystalline powder of adenosine phosphate with solvent-free fullerene powder to produce a first mixture; shearing the first mixture in a shearing mill while maintaining temperature below 55° C. to produce a covalently reacted first mixture; combining a dry crystalline amino acid powder with solvent-free fullerene powder to produce a second mixture; shearing the second mixture in a shearing mill while maintaining temperature below about 54° C. to produce a covalently reacted second mixture; combining the first mixture with the second mixture to create a heterogeneous combination of each; adding about 20 parts water to one part of the combined mixture; irradiating the aqueous mixture with ultrasound at 200 watts and 40 kilohertz for about 20 minutes to homogenize unlike fullerene derivatives by inductive van-der-Waals intercalation to produce homogenized ATP-fullerene pivot amino acid-fullerene conjugates; mixing ATP-fullerene pivot amino acid-fullerene into water containing about 10% glycerol to produce an ATP-fullerene pivot amino acid-fullerene conjugate solution; and mixing the ATP-fullerene pivot amino acid-fullerene solution with a predetermined substrate to form an ATP-fullerene pivot amino acid-fullerene formulation.

Some embodiments are described in detail with reference to the related drawings. Additional embodiments, features, and/or advantages will become apparent from the ensuing description or may be learned by practicing the invention. In the FIGURES, which are not drawn to scale, like numerals refer to like features throughout the description. The following description is not to be taken in a limiting sense but is made merely for describing the general principles of the invention.

### BRIEF DESCRIPTION OF DRAWINGS

Preferred embodiments of the invention will now be described, by way of example, with reference to the accompanying drawings, in which:

FIG. 1 is an illustration of the chemical structures of two essential amino acids, L-Lysine and L-Arginine, in accordance with the teachings of the present invention;

FIG. 2 is an illustration of the reversible reaction of ATP to ADP at physiological pH.

FIG. 3 is an illustration of the reaction of ATP with fullerene at neutral pH to form ATP-fullerene.

FIG. 4 is an illustration of ATP-fullerene with multiple ATP functional groups at physiological pH.

FIG. 5 is an illustration of the reaction of amino acid L-Arginine with fullerene to form Arg-fullerene.

FIG. 6 is an illustration of the reaction of amino acid L-Lysine with fullerene to form Lys-fullerene.

FIG. 7 is an illustration of the reaction of two different amino acids L-Lysine and L-Arginine with C70 fullerene to form Lys-Arg-C70.

FIG. 8 is an illustration of an electrodynamic biaxially pivoting fullerene pivot molecules showing dynamic out-of-plane axial rotation and in-plane twist.

FIG. 9 is an illustration of the closed pincer position of electrodynamic biaxially pivoting fullerene pivot molecules showing proximal counter-ionic functional groups.

FIG. 10 is an illustration of an irradiated treatment area containing at least one oscillating pincer of electrodynamic biaxially pivoting fullerene pivot molecules.

FIG. 11 is an illustration of the displacement and eviction of viral assembly proteins and nucleotides by EPF.

FIG. 12 is a block flow diagram of a method of synthesis of a pivoting electrodynamic fullerenes which contain ATP-fullerene and Lys-Arg-fullerene.

FIG. 13 is a flowchart showing steps to prepare human blood plasma for injection of EPF.

FIG. 14 is an illustration of experimental data for radio frequency signal power attenuation with frequency as applied to EPF.

FIG. 15 is an illustration of experimental mass spectrograph data for ATP derivatized C60.

FIG. 16 is an illustration of experimental mass spectrograph data for L-arginine derivatized C60; and

FIG. 17 is an illustration of experimental mass spectrograph data for EPF.

Embodiments are described in detail with reference to the related drawings. Additional embodiments, features, and/or advantages will become apparent from the ensuing description or may be learned by practicing the invention. In the FIGURES, which are not drawn to scale, like numerals refer to like features throughout the description. The following description is not to be taken in a limiting sense but is made merely for describing the general principles of the invention.

### DETAILED DESCRIPTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations to the drawings, steps, methods, or processes, depicted therein without departing from the spirit of the invention. All these variations are within the scope of the present invention. Hence, specific structural and functional details disclosed in relation to the exemplary embodiments described herein are not to be interpreted as limiting, but merely as a representative basis for teaching one skilled in the art to variously employ the present embodiments in virtually any appropriate form, and it will be apparent to those skilled in the art that the present invention may be practiced without these specific details.

Various terms used in the following detailed description are provided and included for giving a perspective understanding of the function, operation, and use of the present invention, and such terms are not intended to limit the embodiments, scope, claims, or use of the present invention.

A composition of partly exposed hydrophobic fullerene cores can be provided with abutting rotational and pivoting carbon faced surfaces, where these fullerene cores are provided with at least one derivatized adenosine (mono, di, tri) phosphate, and desirably also an equal proportion of derivatized amino acids. Anti-viral methods of treatment incorporating this composition as a medicament are directed at the prevention, treatment, and cure of diseases such as influenza (flu), Alzheimer's Disease, as well as virulent virus infections that may lead to some types of cancer. Both C60 and C70 fullerenes may be used, in various embodiments. Pivoting biaxial electrodynamic fullerene compositions can be used to treat budding virus infections by disrupting the electrostatic replication environment in their buds or pockets within the membranes of infected cells.

One aspect of the composition of the present invention is a phosphate fullerene derivative provided with phosphorus with a +5 oxidation state within a desired multiplicity of pendant functional groups containing phosphate or (PO₄). This phosphate component acts to distribute an analog to adenosine triphosphate (ATP) into cells as prophylactic molecules to disrupt the geometric angular assembly of HSV and other budding viral capsids. It is conceived that viral and cancer disease states that rely on the recruitment of cellular ATP can be mitigated by the careful design of this chemical structural geometry, to interrupt and deter electrostatic symmetry or electrostatic self-assembly by means of a dynamic change in the electrostatic environment and cytosol medium in which viral replication takes place.

The provided ATP-fullerenes are analogs of ATP that can contribute to cell homeostasis while conferring distorting electric fields to the stable electric environment needed to replicate nearly any known virus. These dynamic pivoting antiviral molecules are especially targeted to avoid chronic neurological pathologies based on viral recruitment of ATP in Alzheimer's disease, as well as to significantly reduce the pathology of budding virus pandemics. Inhibition of herpes simplex virus HSV-1 by penetration of these phosphate fullerenes into the endoplasmic reticulum of cellular mitochondria will prevent the correct angular fitting of viral proteins to form HSV capsids. The ATP-fullerenes attract viral proteins to create incorrect spacing and geometry of charges in the viral assembly process to form mismatched regions that will no longer align to mate with partner capsid proteins to allow the formation of the mature virus.

In a related aspect, the fullerene phosphates are configured to function in the manner of ATP by the reversible loss of a pendant phosphate group to form fullerene-AMP (adenosine monophosphate) pendant groups, while allowing the cell to survive and operate the typical electron transfer pathways used by native cellular ADP and ATP used to respire and sustain life.

Advantageously, at least some of the poly-phosphorylated fullerene molecules express geometric localization of polyphosphates to one pivot molecules at one face or hemisphere of the substantially spherical carbon molecular cage of the fullerene structure, to enable a hydrophilic face directed at mitigating reactive oxygen species (ROS) at the interface between the endoplasmic reticulum (ER) of the mitochondrial cell membrane and the cytosol or water based fluids abutting the ER, while allowing one region of the fullerene core to attach to a cell lipid membrane or a microtubule used in cellular transport. This may avoid molecular damage of cell structures through oxidative stress that can leave cells susceptible to invasive pathogens.

In an embodiment, the composition of the medicament includes additional fullerene molecules that express pendant amino acids, to enable a hydrophilic face directed at reactive oxygen species (ROS) at the interface between the endoplasmic reticulum (ER) of the mitochondrial cell membrane and the cytosol or water-based fluids abutting the ER. The amino-fullerenes function to deactivate viral capsids by binding with them to provide both an anchor and a more permanent seal to prevent infection by the release of viral contents to the cell and the cell nucleus.

In one aspect, the fullerenes destabilize and destroy mycobacteriophages, and thereby assist commensal fungi or bacterial organisms normally in human tissue from indirectly performing genetic DNA methylation via microbial defense mechanisms that release toxins as part of their normal viral toxification mechanisms when being infected by virus particles. This significantly helps to reduce and avoid the creation of improperly folded proteins such as tau and beta amyloid associated with neurological pathologies found in Alzheimer's disease and may reduce the likelihood of environmentally assisted mutagenicity that may in some cases lead to cancer.

In a related aspect, a scissoring action of fullerene pivot molecules containing both positively charged amino fullerenes and negatively charged adenosine phosphate fullerenes act to pierce amyloid plaque salt bridges and to unfold misfolded proteins, thereby allowing these to be more easily disentangled, dispersed, and cleared from the brain extracellular environment as mobile detritus.

Another aspect is the provision of fullerene phosphates and amino fullerenes to cooperatively treat and reduce the spread of budding viruses such as influenza and HSV. One function of the derivatized fullerenes includes the protection of undefended positive ends of dynamic actin filaments used by herpes simplex virus particles (HSV) to invade the cell, and then at a later stage of the viral reproductive cycle, to leave the cell using the negative ends of the microtubule after replication. Fullerene polyphosphates prepared with points of negative charge at their distal spikes are provided to bind to the same positive charged regions of the actin filaments used to transport proteins and glucose into the cell, where the HSV also arrives. If this prevention by displacement fails, then an amino-acid-fullerenes are provided to bind to the same negative charged regions of the actin filaments used to transport proteins and wastes out of the cell.

In another embodiment, pivoting electrodynamic fullerenes diffuse to virus bud cavities, where they then charge-attract and bind with replicating viral components and capsids, especially where the viral proteins have not yet completed the formation of the capsid enclosure, to denature the assembly process, and disrupt the ambient electric fields of electrostatic charge maintenance to allow eviction of the invasive proteins and virus particles by normal diffusion.

In a related aspect, the diffusion eviction process of the electrodynamic fullerene moiety is amplified with the assistance of the application of concentrated radio waves broadcast to the infected person, or an AC electric signal applied to the targeted tissues to treat the targeted organ of infection and inflammation. This action has the effect of magnifying the torsional twist about abutting fullerene centers in any pivot molecules, as well as inducing an out of plane twist motion among or between the oppositely charged ionic functional groups of these fullerene pivot molecules. These dynamic motions are associated with local directional electric field changes in the immediate vicinity of the pivoting derivatized electrodynamic fullerene pivot molecules, thereby destroying those electrostatic field conditions that are necessary to preserve the structural integrity of viral structures and disabling the static conditions needed to promote viral component self-assembly at membrane-based buds providing viral molecular self-assembly platforms. Such energy can be delivered by RF radiation or by electric waves at a frequency of about 8 GHz to about 10.6 GHz.

Recently has it become apparent that these virus-inspired cytoskeleton changes in cells infected with herpes virus, also promote the cell to transform into a cancerous cell. This is because the cells are made to counteract normal growth, after they have been genetically altered to “obey” the virus, thereby causing cancer and the spread of cancer, long after the virus has left. The application of the compositions herein allows the treatment of viral infection to reduce a significant risk of cancer or propagation of cancerous cell growth. Because other neuropathies such as multiple sclerosis (MS) and Alzheimer's disease (AD) also implicate viral infection as a causative agent, the present invention is to be directed at MS, AD, and any other diseases having certain types of virus particles as part of their etiology and disease progression, especially when these virus particles require utilization of cellular ATP, and may be treated by a molecular masquerade of fullerenes that have been decorated or derivatized with ATP.

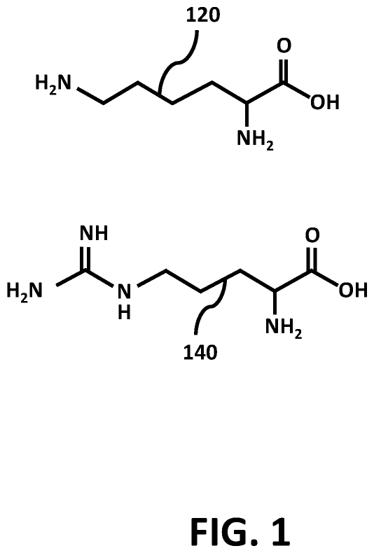

Referring now to the drawings wherein like elements are represented by like numerals throughout, FIG. 1 is an illustration of the chemical structure of one molecule of amino acid L-Lysine (Lys) 120, at neutral pH (pH≈7.0) and the chemical structure of one molecule of amino acid L-Arginine (Arg) 140, at neutral pH. Both Lys 120 and Arg 140 are white crystalline solids at room temperature. Any amino acid may be used to construct a positive charged pendant group, and any other substituent having positive charge may be considered for use in like manner as starting materials to make the positive half of a pivot molecular pair such as exemplified by the amino-acid-fullerenes. L-Lysine is, however, the primary raw material conscripted in the greatest amount by the herpes simplex virus (HSV) particles from the cellular environment to duplicate itself. L-Arginine is well known to be associated with raw materials conscripted and redirected for the unregulated growth, and propagation, of cancer cells that have been detrimentally reprogrammed by virus particles. These two amino acids, Lys and Arg, are therefore related to the cellular deficits associated with disease states. Since both HSV infection and cancers arising in great part from genetic reprogramming are related, L-Lysine is therefore an amino acid of primary functional importance, and L-Arginine is of secondary importance in the raw materials and functionality required for the purpose of the amino-acid-fullerene treatment.

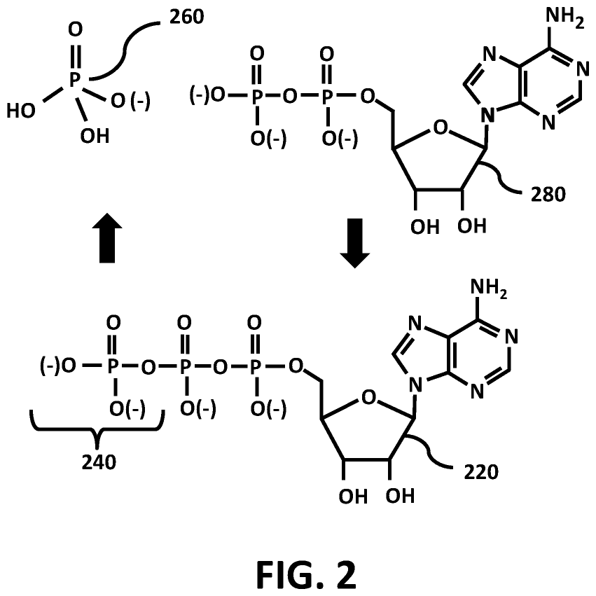

FIG. 2 is an illustration of the structure of a molecule of adenosine triphosphate (ATP) 220 having a terminal phosphate group 240. ATP 220 undergoes reaction with one molecule of water (not shown) to release the terminal phosphate group 240 as a phosphate ion 260, as shown by the direction of the upward pointing black arrow. This leaves a shortened molecule with one less phosphate group, i.e., adenosine diphosphate (ADP) 280. This reaction is reversible, as indicated by the downward direction of the black arrow, to again form ATP 220. In like manner, another phosphate group may reversibly leave from ADP to form adenosine monophosphate or AMP in the manner generally understood (but not shown here) to be part of the cycle of cellular respiration associated with the electron charge transfer process in cellular biology. The cellular respiration processes are substantially performed at the mitochondrion of the cell. Each of the phosphate groups that are part of ATP 220, phosphate ion 260, and ADP 280 are shown to be deprotonated or having a negative charge (−) in accordance with the state of physiological pH within the cell, to indicate that the conditions are favorable for the reversible addition or loss of phosphate groups required for cellular respiration. The atoms of the phosphorus in ATP 220, ADP 260 retain a chemical oxidation state value of +5 that is not known to alter as these chemical processes reversibly proceed during normal cellular respiration.

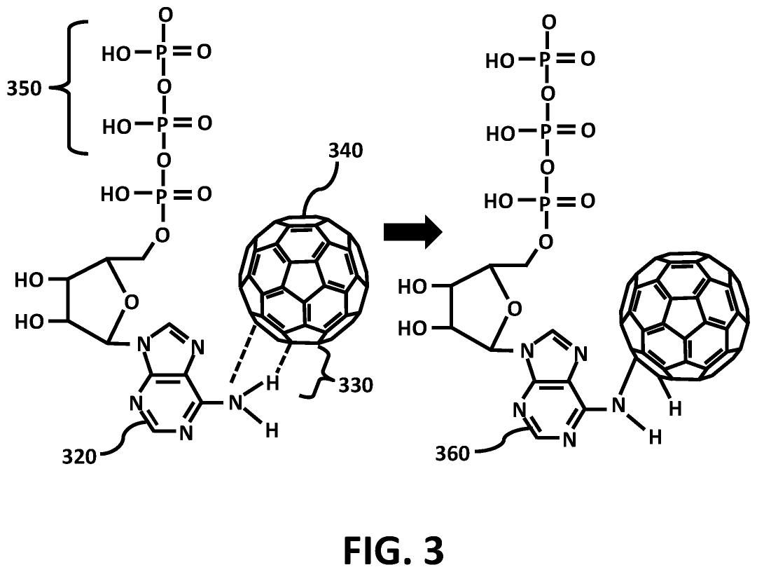

FIG. 3 is an illustration of the reaction of a molecule of ATP 320 with a molecule of fullerene 340 at neutral pH to form ATP-fullerene. Region 350 of the ATP molecule is highlighted to indicate that this portion of the phosphate 320 may be replaced by equivalent adenosine monophosphate (AMP), or adenosine diphosphate (ADP), while not materially altering the nature of the initial hydrogen bonding and subsequent covalent reaction at the region of the primary amine 330 with fullerene 340 in each of these cases. The creation of ATP-fullerene is indicated by the direction of the black arrow to show this final reaction product. ATP-fullerene will, under physiological pH (pH≈7.4) conditions, reversibly convert to ADP-C60 and then to AMP-C60 in the manner generally understood among the reversible chemical transformations of AMP, ADP, and ATP as part of the electron transfer cycle in cellular respiration.

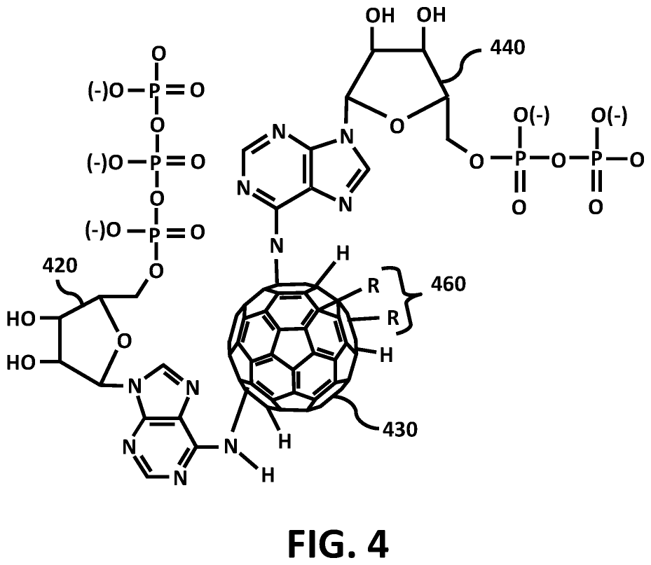

FIG. 4 is an illustration of a multiply derivatized ATP-C60 molecule at physiological pH (pH≈7.4) with an ATP functional group 420, and two other molecularly identical ATP functional groups 460 herein represented by the letter R. A fourth functional group has reversibly lost one phosphate group to from an adenosine diphosphate 440. The ATP-fullerene molecule takes part in the respiration of the cell, especially at the mitochondrion, while the core fullerene 430 is also serving to act as a powerful antioxidant. The fullerene core 430 is well known to be able to collect free radicals such as hydroxyl free radicals, and combine these to form harmless hydrogen peroxide, which can be removed by the cell as waste. It is notable that even when ATP-fullerene has multiple ATP functional groups disposed at different three-dimensional angles from each other, each phosphate group is still able to assist in reversible loss of phosphate as part of the cellular respiration cycle. For example, the phosphate groups of ADP 440 are shown disposed at right angles to those of ATP 420. This complex geometry does not impair the ability to accrue or lose phosphate groups in the manner of ordinary ATP used by the cell for respiration and the transfer of chemical energy. ADP functional group 440 is expected to reform into an ATP functional group.

However, unlike conventional ATP, the ATP-fullerene nanoparticle creates a geometric size anomaly when it is incorporated into the regular structure of a virus particle, thereby throwing off the angular dependence and symmetry needed to knit together the seams of the abutting viral protein plates using multiple identical ATP molecules as part of the HSV protective covering. This three-dimensional complexity of ATP-fullerene confers artificial innate immunity to cells against virus particles using intelligent three-dimensional geometric design and constitutes a novel and critical new biological defense function for this nanoparticle.

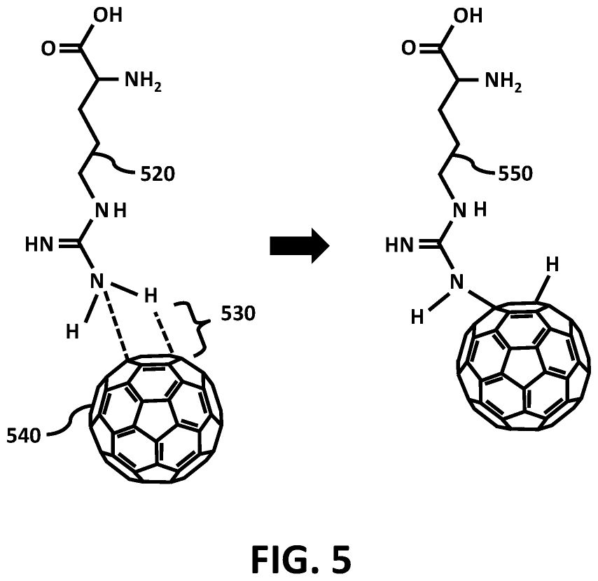

FIG. 5 is an illustration of the reaction of one molecule of amino acid L-Arginine 520, with the fullerene 540, to form an amino acid adduct by hydrogen bonding with the fullerene 540, where the dashed lines indicate the presence of hydrogen bonds 530. On further chemical activation, such as by applied heat or microwave irradiation, this adduct becomes a covalent derivatized Arg-fullerene 550. In like manner, several more molecules of L-arginine can be provided to react with the fullerene 540, where each such addition confers greater water solubility to the resulting Arg-fullerene derivative. Ideally, a pendant amino-acid functional group derivative of the fullerene 540 can achieve a desirable hydrophilic property.

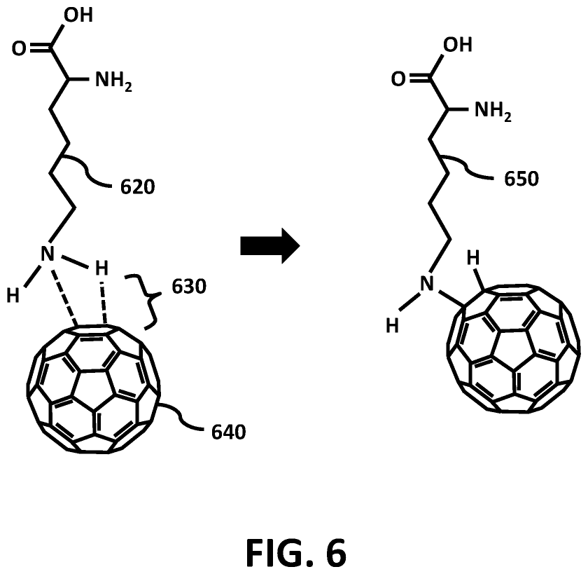

FIG. 6 is illustrated the reaction of one molecule of amino acid L-Lysine 620, with fullerene 640, to form an amino acid adduct by hydrogen bonding with fullerene 640, where the dashed lines indicate the presence of hydrogen bonds 630. On further chemical activation, such as by applied heat or microwave irradiation, this adduct becomes a covalent derivatized Lys-fullerene 640. In like manner, several more molecules of L-Lysine 620 can be provided to react with the fullerene 640, where each such addition confers greater water solubility to the resulting Lys-fullerene derivative. Ideally, a pendant amino-acid functional group derivative of the fullerene 640 can achieve a desirable hydrophilic property.

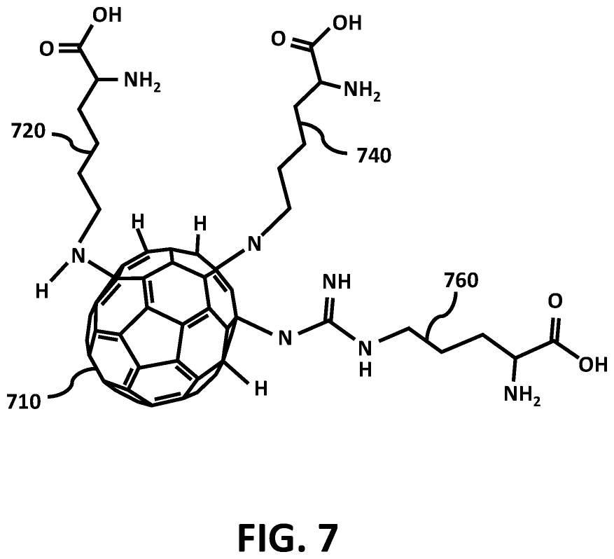

FIG. 7 is an illustration of a lysine arginine C70 fullerene molecule. The lysine arginine C70 molecule contains a 70-carbon atom cage structure of the fullerene functional group 710 and two amino acid groups of L-Lysine 720, 740 and one group of L-Arginine 760. It is understood that amino acid functional groups other than L-Arginine or L-Lysine may be selected to confer amine functionality as one constituent of a pivoting fullerene according to the intent of this invention, wherein the amine groups are highly able to target viral RNA as well as neoplastic (cancerous) DNA. When both L-Lysine and L-Arginine are available in excess for the method of synthesis of Lys-Arg-C70 , there will be a mixture consisting of a plurality of differing ratios of either L-Arginine or L-Lysine functional groups attached to any given molecule of Lysine-Arginine-C70 . It is understood that the core fullerene can be another fullerene such as C60 , however here it is shown that C70 is also able to accommodate pendant amino-acid derivatives on the core fullerene. The result of having a more massive core fullerene of 70 carbons, as well as having more surface area on the core fullerene, is to allow the change of core fullerene molecular mass and therefore provide a change of electromagnetic frequency of activation of the resulting pivot conjugate of the present invention. By increasing the mass, the duration of pivoting of any van-der-Waals inductively attracted fullerene to a partner derivatized fullerene by electromagnetic activation and attenuation of such energy, must allow greater time of activation to move the mass about the pivot. The resultant deformations, being rolling pivots at core fullerene to core fullerene at inductive surface attraction points will require more energy to move a greater mass; this results in a lower frequency of inductive reactance to electromagnetic energy at a desired electromagnetic activation than that of lighter molecular weight moieties made with a C60 fullerene pivot group. This can be useful when different intercalated drugs need to be released at different times at the same point on the targeted organ using two different mass pivot conjugates having relatively different resonant electromagnetic signal attenuation frequencies.

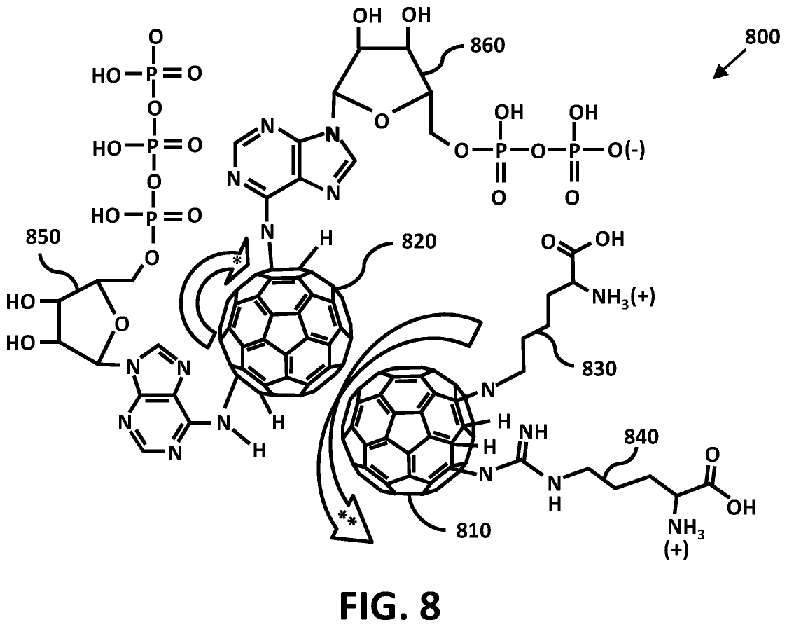

FIG. 8 is an illustration of electrodynamic biaxially pivoting fullerene pivot molecules 800. Core fullerene 810 is bound by van-der-Waals charge induction to core fullerene 820 at an abutting point of contact that rotates as shown by the direction of the curved white arrow marked with one Asterix symbol. At any time, core fullerene 810 may pivot about the abutting point of contact with core fullerene 820 to twist out of the plane of this schematic as shown by the large arrow marked with two Asterix symbols. Both the in-plane rotation and the out-of-plane axial rotation are dynamically changing depending on shifting currents of the cell cytosol or the intercellular matrix in which biaxial molecular pivot 800 is floating or dissolved. Core fullerene 810 is provided with a pendant lysine amino acid functional group 830 and an arginine amino acid functional group 840, as well as at least two pendant hydrogen atoms that are represented by the atomic symbol for hydrogen, ‘H’. Core fullerene 820 is provided with a pendant adenosine tri-phosphate functional group 850, and a pendant adenosine diphosphate functional group 860, where group 850 and group 860 may participate in the electron transfer cycle of cellular respiration in the manner of free molecules of adenosine tri-phosphate (ATP) and in the manner of free molecules of adenosine di-phosphate (ADP), respectively. Core fullerene 820 is provided with at least two pendant hydrogen atoms. Pendant phosphate functional groups 850 and 860 are provided to be recruited by virus proteins in the manner of ATP or ADP for the purpose of self-assembly of the viral nucleotides, as well as for the purpose of self-assembly of the viral protein components normally used to replicate a virus using cellular molecules and the cellular molecular constituents. However, by being so recruited, the physical obstruction or steric hindrance of any of a multiplicity of functional groups such as represented by functional groups 830, 840, 850, 860 will interfere with the further assembly of the virus particle.

Additionally, the presence of positive charges on the amine groups of pendant amino acids 850, 860, or like amino acid functional groups as shown in FIG. 7, will alter the electrostatic environment of the virus constituents to create an electrodynamic environment of changing charge densities and positive charge locations leading to conditions that are unfavorable to the viral self-assembly process, because of the biaxial twist and rotation as well as the steric hindrance of any number of core fullerene molecules such as 810, 820.

Moreover, the presence of negative charges on the phosphate groups of pendant adenosine phosphate molecules 850, 860, or like fullerene phosphate functional groups as shown in FIG. 3 and FIG. 4, will alter the electrostatic environment of the virus constituents to create an electrodynamic environment of changing charge densities and negative charge locations leading to conditions that are unfavorable to the viral self-assembly process, because of the biaxial twist and rotation as well as the steric hindrance of any number of core fullerene molecules such as 810, 820.

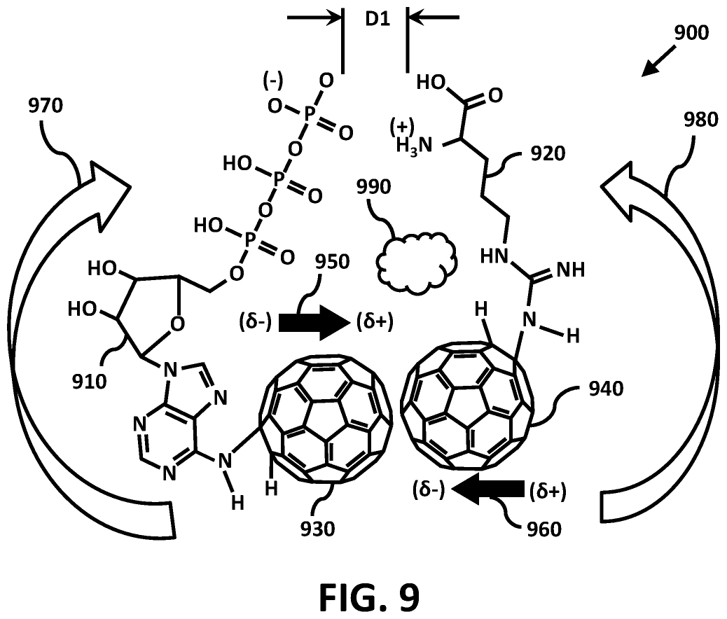

FIG. 9 is an illustration of the substantially closed pincer position of electrodynamic biaxially pivoting fullerene pivot molecules 900, provided with adenosine triphosphate functional group 910 and amino acid functional group 920 serving as a molecular pincer or gripper in the gap region indicated by distance D1, having a dimension of about 1 nanometer or less in the configuration of 900. Partly exposed hydrophobic core fullerenes 930, 940 are provided with mutually abutting rotational and pivoting carbon faced surfaces to allow in plane rotation and out-of-plane rotation about the region of their mutual abutment. Hydrophobic core fullerenes 930, 940 induce mutually attractive London Dispersion Forces 950, 960. Core fullerene 930 obtains a partial positive charge in the direction of attraction 950 towards the core fullerene 940. Simultaneously, the fullerene group 940 obtains a partial negative charge in the direction of attraction 960 towards the core fullerene 930.

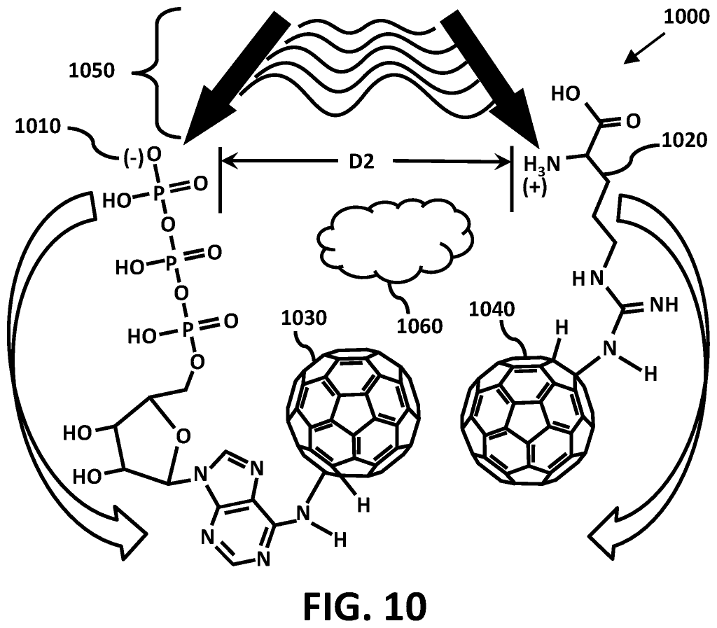

Core fullerene 930 obtains a partial negative charge in the region away from the direction of attraction 950 towards core fullerene 940. Simultaneously, the core fullerene 940 obtains a partial positive charge in the region away from the direction of attraction 960 towards core fullerene 930. This process of electrostatic attraction by dispersed partial electronic charges of opposing type is generally known and well described in the scientific literature as the van-der-Waals effect. The pivoting electrodynamic fullerenes relies on van-der-Waals attraction 950, 960 to implement the pivoting function of abutting core fullerenes exemplified by representative core fullerenes 930, 940 in the manner of two abutting ball-bearings that are constructed using nanometer-sized molecules. The van-der-Waals attractive forces 950, 960 serve as self-supporting attractive anchors for the core fullerenes 930, 940 to permit a forceps or pincer type of fulcrum function where the pair of large arrows 970, 980 show the directions used to bring together the molecular armatures provided by the adenosine tri-phosphate derivative 910, and the exemplary arginine amino acid derivative 920 into proximal distance indicated by D1. Under cytosol conditions of physiological pH, adenosine triphosphate group 910 obtains a negative charge at a terminal phosphate group, and arginine amino acid group 920 obtains a positive charge at the amine group, both of which opposing electrostatic charges allow each to become reversibly attracted to each other or to become reversibly attracted to counter-opposing charges in viral proteins or viral nucleic acids. Optionally, the collective structures of the electrodynamic biaxially pivoting fullerene derivatives composition are attracted to and carry a therapeutic molecular drug cargo indicated by the intercalated substance 990, being an antibody, an anti-cancer gallium ion Ga3+, or other therapeutic substance that is to be delivered to an intended organ and cellular site such as a tumor or a cancerous growth that can be associated with pathogenic viral infection as an initiator of the tumor or cancer. Extraction of the delivered therapeutic cargo 990 is provided by the widening of distance D1, such as when the surrounding electronic conditions permit. For example, when the negatively charged phosphate group on pendant phosphate armature derivative 910 becomes attracted to a positive surface charged cell membrane lipid such phosphatidyl serine. As another example, when the positively charged amine group on pendant amino-acid armature 920 becomes attracted to a negative surface charged cell membrane lipid such as phosphatidyl choline. In addition, other factors can influence the electrostatic field near or abutting to electrodynamic biaxially pivoting fullerene pivot molecules 900, such as the provision of electromagnetic irradiation as illustrated in FIG. 10.

FIG. 10 is an illustration of the irradiation of a treatment area 1000. The treatment area contains at least one substantially open pincer position of two electrodynamic biaxially pivoting fullerene molecules 1000 which oscillate upon being irradiated. One of the fullerene pincer molecules 1000 is provided with an adenosine triphosphate functional group 1010 and the other with an amino acid functional group 1020 serving as a lever and a molecular pincer or gripper in the gap region indicated by distance D2, having a dimension of about 1 nanometer or greater in the configuration of pivot 1000. Partly exposed hydrophobic core fullerenes 1030, 1040 are provided with mutually abutting rotational and pivoting carbon faced surfaces to allow in plane rotation and out-of-plane rotation about the region of their mutual abutment that function as a fulcrum by means of van-der-Waals forces shown in FIG. 9. The extension or widening of gap D2 in the direction of two curved large white arrows is facilitated by the application of electromagnetic irradiation 1050 with propagation indicated by wavy lines moving in the direction of two large black arrows, wherein this irradiation is preferably in a microwave region that is away from a dipole resonant frequency of water so as not to damage or denature the healthy cellular components used to sustain the living processes of the cell. Electromagnetic energy 1050 serves to energize and reversibly actuate a scissoring of the molecular armatures 1010, 1020 at the resonant frequency of the electrodynamic biaxially pivoting fullerene pivot molecules 1000. This thermo-mechanical actuation serves to generate local heating to facilitate the release of therapeutic constituent 1060 being an exemplary drug, gallium Ga3+ ions, antibody, mRNA, or a vaccine antibody for targeted delivery to the environment of the organ being treated for a viral infection, cancer, or other pathogen.

The electromagnetically actuated electrodynamic biaxially pivoting fullerene molecules 1000 are provided with proximal counter-ionic functional groups 1010, 1020 which bond to and stretch apart the angularized viral proteins and serve to displace and distort nucleic components growing from their protected positions against the inner membrane wall bud of the cell that has been pinched off and overtaken by the micromachinery of replicating virus. The application of a dynamically changing electric environment provides the pivoting antiviral fullerene composition the ability to lyse, scissor, disrupt, and ‘chop’ viral replication platforms and cancers. This serves to disrupt their electrostatic stasis, thereby creating a dynamic viral disassembler. The irradiation amplitude and frequency of the irradiation 1050 actuates the molecular armatures 1010, 1020 to reversibly cycle the stretched distance of the gap D2 between a negative charged phosphate group 1010 and a positive charged amine group 1020. This action also allows the controlled release of the optional drug constituent 1060, and the deactivation of virus or virus components as well as the cell lysing of cancer cells provided by the electromagnetically induced rotation and twist of the pivoting fullerene molecule 1000. The region of the irradiation 1050 defines the treatment area that contains a multiplicity of fullerene molecular pivots 1000. The region of the irradiation 1050 can be targeted to a single tumor, or it can be a larger targeted region such as an organ or portion of a limb, or in some embodiments, such as for example to treat a diffuse cancer such as leukemia, it can be the entire body of a person, in accordance with the intent of the present invention.

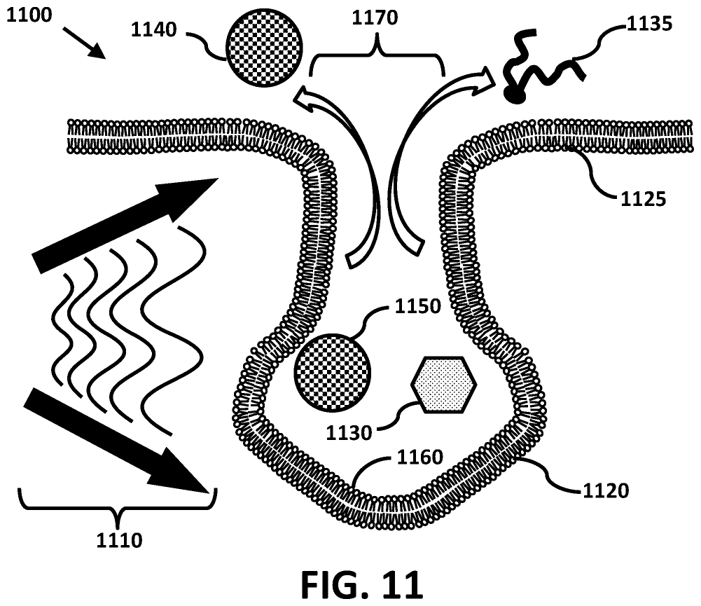

FIG. 11 is an illustration of electromagnetic radiation 1110 applied to a virus induced membrane bud 1100. Electromagnetic radiation 1110 is applied to the cell region of an enteric pocket or bud 1120 formed out of the constituents of the cell phospholipid bilayer 1125 by a virus. The enteric bud 1120 has the purpose of shielding the viral proteins 1130 and viral nucleotides 1135 from local changes in the electric field to allow self-assembly of a multiplicity of components of the virus using local attraction and binding to diffusing cellular molecules and materials delivered by the cell, such as adenosine tri-phosphate (ATP). The injection of the scissoring fullerene pivot molecules 1140, 1150 are provided with least one functional group chemically similar to ATP to become bound to the viral structures of a multiplicity of viral particles 1130 that have clustered at the interior walls of the membrane bud 1160. The unstable electric fields generated by the pivoting and out-of-plane twist of Electrodynamic Antiviral Fullerene pivot molecules 1140, 1150 causes displacement and eviction of viral replicant assembly proteins and nucleotides 1135 from the membrane bud 1120 as shown by the direction of the two curved white arrows at the lipid membrane bud entrance 1170. The effect of local changing electric fields around the electrodynamic biaxially pivoting fullerenes 1140, 1150 is to provide thermo-mechanical energy by the induction of heat and displacement via the directed electromagnetic radiation 1110. Applied electromagnetic radiation 1110 in the form of gigahertz radio waves has the direction of propagation shown by two large black arrows within the region shown with wavy black lines. The electromagnetic radiation 1110 can be a millimeter radio wave of the microwave band that is not at a water dipole resonant frequency or wavelength, to avoid aqueous cellular cytosol heating or thermal damage to cellular structures.

The electromagnetic radiation 1110 can also be optionally introduced to the virally infected region or a tumor as an electrical voltage in the form of, without limitation, sinusoidal, square, or sawtooth waves. Electromagnetic radiation 1110 is desirably resonant with the structure of the electrodynamic biaxially pivoting fullerene composition 1140, 1150, thereby enhancing and further energizing the natural biaxial pivoting and changing local electric fields of these molecules. The electromagnetic radiation 1110 provides more energy than that associated with random thermal vibrations, so that the electrostatic environment is disrupted sufficiently to prevent the viral assembly and replication conditions within membrane buds 1120 by removing the state of unchanging electrostatic fields needed to self-assemble virus particle components such as representative viral proteins and nucleotides 1135.

The electromagnetic radiation 1110 can also be light waves between the wavelengths of about 800 nm to about 1000 nm where living tissues are translucent. It is to be understood that any combination of the RF, light, and electrical types of irradiated waves can be applied to the electrodynamic biaxially pivoting fullerene composition 1140, 1150, in any sequence and in any combination whatsoever to help energize the therapeutic treatment using this composition.

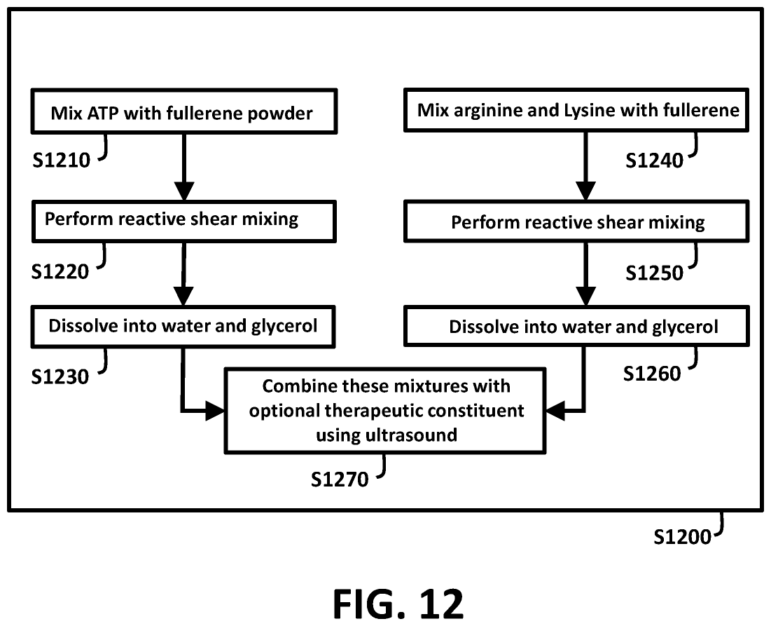

FIG. 12 is a flow chart showing an exemplary method S1200 of synthesis of electrodynamic biaxial pivoting fullerenes (EPF). The EPF composition is a mixture of ATP-fullerene and Lysine-Arginine-fullerene. In a step S1210, an excess of dry crystalline powder of adenosine triphosphate is added to solvent-free and dry crystalline fullerene in a nominal ratio of 1 mole fullerene to 2 moles of ATP. In a step S1220, the combined mixture of dry powders is placed into a shear grinding mill and allowed to react at greater than 1000 per second shearing rate below 55° C. for about 15 minutes. Because this reaction takes place in air, the temperature may not exceed about 60° C. or be operated in the grinding process for a longer time, because of thermal degradation and oxidative decomposition of the non-fullerene portion of the reactants. A lower limit of one and an upper limit of about 3 to 4 ATP derivative functional groups can easily be bonded to a core fullerene in this process, however one group is sufficient to allow the bare carbon face of the core fullerene to perform a pivot function. Some excess ATP may become entrapped or intercalated between the produced ATP-fullerene molecules. However, these do not otherwise reduce the efficacy of the product. In a step S1230, the reacted products of step S1220 are dissolved into water with at least 10% glycerol solvent to make a dispersion, where the solvents are selected to expedite the desired delivery of medicament in the finished product mixture.

In a step S1240, an excess of dry crystalline powder of L-lysine and L-arginine are added to solvent-free and dry crystalline fullerene in a nominal ratio of 1 mole fullerene to 1 mole of lysine and 1 mole of arginine. Any amino-acid capable of providing an amine functional group near or at the terminal end of the amino-acid, can provide the desired positively charged molecular armature. However, the selection of two different types or lengths of amino-acid as pendant fullerene functional groups helps to ensure a complex electrodynamic environment for this ingredient of the composition.

In a step S1250, the combined mixture of dry powders is placed into a shear grinding mill and allowed to react at greater than 1000 per second below 55° C. for about 15 minutes. Because this reaction takes place in air, the temperature may not exceed 60° C. or be operated in the grinding process for a longer time, because of thermal degradation and oxidative decomposition of the amine reactants. A desired lower limit of one or two amino acid functional groups, with an upper limit of about 3 to 4 amino acid functional groups can easily be achieved as derivatives to the core fullerene in this process. Some excess amino acid molecules will become entrapped or intercalated between Lysine-Arginine-fullerene molecules, however these do not otherwise reduce the efficacy of the product, providing enough bare carbon fullerene core remains present to enable the pivoting action of the core fullerenes in the final composition. In a step S1260, the reacted products of step S1240 are dissolved into water with about 10% glycerol solvent, where the solvents are selected to expedite the desired delivery of medicament in the finished product mixture.

In a step S1270, the Lysine-Arginine-fullerene solution and the ATP-fullerene solution are combined in a mixer equipped with ultrasonic actuation. The purpose of the ultrasonic irradiation is to allow inter-dispersion of unlike fullerene derivatives to create the desired hybrid fullerene pivot molecules in suspension. At this point, an optional desired medicament may be added into this mixture. This medicament can be driven into and between the unlike derivatized fullerene molecules to confer enhanced transport into cells, using any physical method of delivery. Exemplary medicaments to be incorporated as a payload to the electrodynamic fullerene pivot molecules may include a drug, multiple drugs, nutraceutical, nootropic, senolytic, anti-cancer, other types of derivatized fullerenes, and any combination thereof without limit, when the dosage is used to cure or prevent disease.

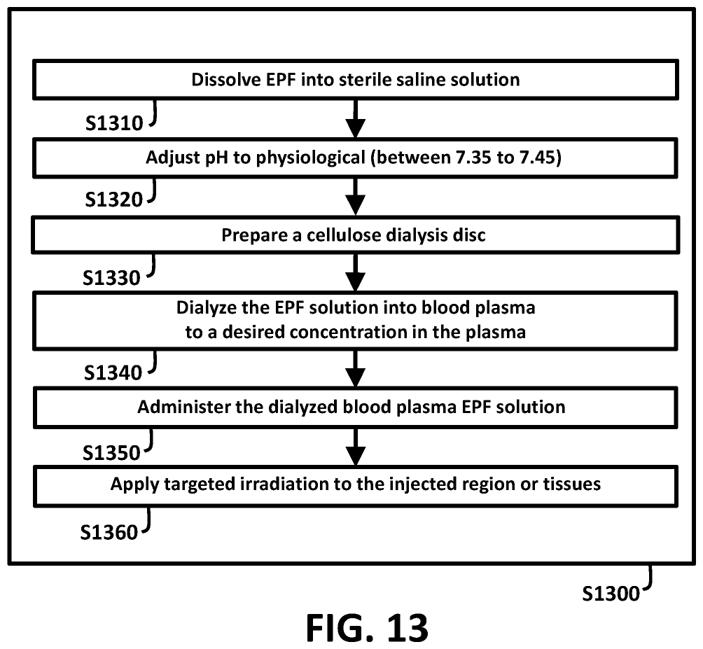

FIG. 13 is a flowchart showing steps of an exemplary method S1300 to prepare and intravenously administer a composition of electrodynamic fullerenes in blood plasma followed by local irradiation. In a step S1310, the electrodynamic fullerene composition is dissolved into sterile saline solution. In some embodiments ultrasound and/or mechanical stirring is employed until the solid particulates are dispersed, indicated by their visible disappearance. The fullerene electrodynamic composition can be produced, for example, by the method outlined in FIG. 12. If necessary, in a step S1320 the pH of the saline solution S1310 is adjusted to a physiological pH from between about 7.35 to about 7.45.

In a step S1330, a cellulose dialysis disc having a molecular weight cutoff of 3500 Daltons, for example, is rinsed with distilled water to remove any packaging glycerol. An exemplary 33 mm disc is the Spectra/Por™ RC, manufacturer number 132488, provided by Repligen at 18617 South Broadwick Street, Rancho Dominguez, Calif. 90220, USA.

In a step S1340, the aqueous EPF solution is dialyzed, in a cellulose dialysis bag for example, into blood plasma to obtain the desired concentration of EPF in sterile blood plasma at a physiological pH. By passing through the dialysis filter, potential impurities greater than 3500 Daltons as well as possible dust is removed. This composition is then transferred to a substrate, which can be a predetermined mixture of other antiviral medicaments, amino acid derivatized fullerenes, or optional preselected polyphenols desired to complete the intended formulation or serving. Ideally, these materials are substantially water soluble or water dispersible, and can be driven into the spaces between ATP-fullerene with ATO-LYS-ARG pivot molecules while mechanically stirring and using ultrasound applied to the combined mixture at, for example, about 200 watts and about 40 kilohertz for about 20 minutes.

In a step S1350, the sterile dialyzed blood plasma EPF solution is administered, for example by injection or intravenous (IV) drip to the patient as specified by an attending physician. A drip rate may be adjusted by an infusion pump to vary depending on the prescribed dosage and concentration, which can relate to the body weight of the patient as well as the severity of the microbial load to be addressed, which has been determined in the patient.

In step a S1360, electromagnetic radiation of, for example, 10.6 gigahertz of radio frequency (RF) is applied to the region or tissues that are the target of the drug release therapy or that are the target of localized tumor treatment. The function of cell lysis is provided by the RF energy supplied to the pivoting electrodynamic biaxial fullerenes to enable their scissoring which is used to actuate the EPF fullerene composition for the purpose of substantially destroying the tumor or cancer cells, or for sterilizing the local region of infected tissues.

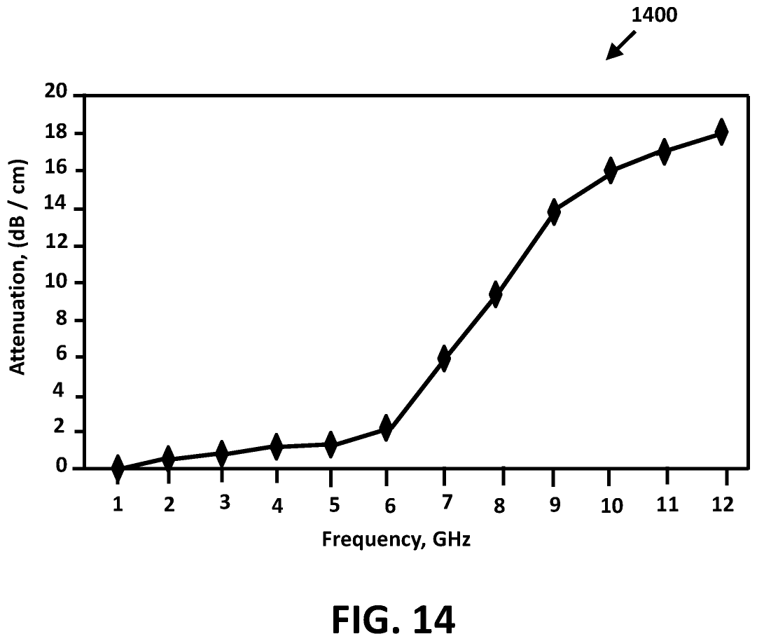

FIG. 14 is an illustration of experimental data for signal power attenuation with applied electromagnetic frequency of activation. The measured decibel of wave energy reduction per centimeter thickness of material is plotted on the Y-axis. It is assumed a large enough sample was measured so that no edge effects are significant in this measurement. The frequency of energy at which the energy absorption was determined, is plotted along the X-axis. By an increase of mass, the duration of pivoting of van-der-Waals inductively attracted derivatized fullerene to a partner derivatized fullerene by electromagnetic activation and attenuation of such energy must allow greater time of activation to move this mass. Such minor components present in the test mixture attenuate less than about 10 percent of the applied energy from about 1 GHz to about 6 GHz as shown by less than about 2 decibels over this range. The primary deformations, being rolling pivots at core fullerene to core fullerene inductive attraction points, require higher frequencies to oscillate their mass; this occurs at a characteristic frequency of inductive reactance to electromagnetic energy, with an energy attenuation of about 17 decibels at about 10 GHz or greater. International radiofrequency transmission from telecommunications towers presently provides an upper limit of about 10.6 GHz for line of sight microwave telecommunications towers to allow a legal safety limit for human exposure to radio frequencies that are away from those frequencies of electromagnetic activation that are known to cause heating and potential damage of biological tissues. For this reason, the application of the present invention must take safety legal requirements into account. Therefore, the operational limit is set to the current international legal limit of about 10.6 GHz for general human exposure to electrical or radio frequencies, even though the graph of test results indicates a slightly greater frequency can be otherwise more efficient at absorbing the applied irradiation to dynamically activate the derivatized fullerene pivots.

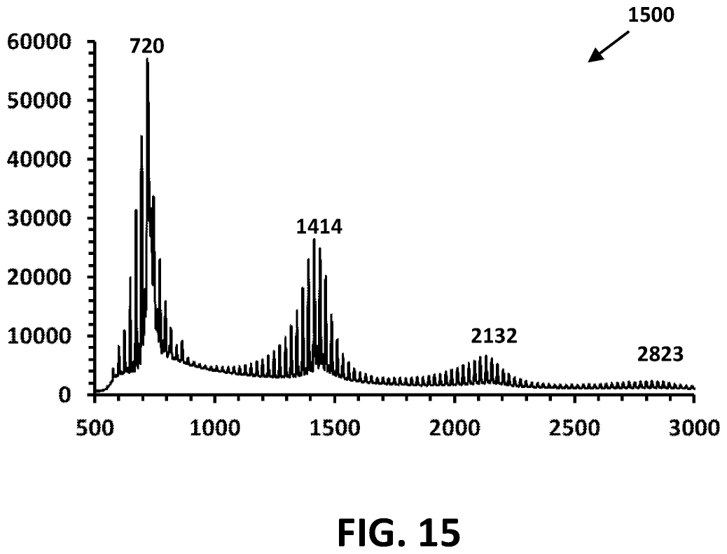

FIG. 15 presents negative mode experimental mass spectrograph data for adenosine triphosphate (ATP) derivatized C60 , where the largest molecular peak at about 720 mass to charge ratio represents the core molecule of C60 after all of the functional groups have been ablated away. The grouping of peaks at mass to charge ratio of about 1414 represents the molecular fragments associated with one adenosine triphosphate group functionalized to one fullerene molecule, as ATP-C60 as the primary reaction product. The grouping of peaks at mass to charge ratio of about 2132 represents the minor amounts of molecular fragments associated with two adenosine triphosphate groups functionalized to one C60 molecule, here designated as (ATP)2-C60 . The grouping of peaks at mass to charge ratio of about 2823 represents the trace amounts of molecular fragments associated with three adenosine triphosphate groups functionalized to one C60 molecule, here designated as (ATP)3-C60.

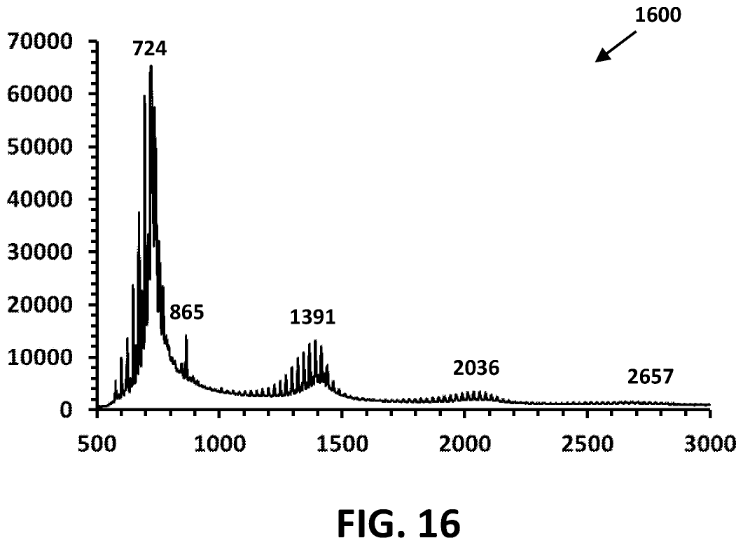

FIG. 16 presents experimental mass spectrograph data for arginine (ARG) derivatized C60 , where the largest molecular peak at about 724 mass to charge ratio represents the core molecule of C60 after all of the functional groups have been ablated away with the exception of four hydrogen atoms. For these hydrogen atoms to be present and incapable of being removed by negative mode mass spectroscopy, it is likely that this molecular fragment of C60 contains covalently bonded hydrogen atoms that are reacted to and face the interior of the carbon cage structure. The single peak at mass to charge ratio of about 865 represents a very characteristic molecular ion fragment associated with an arginine fragment that is still able to remain pendant to the core C60 . The grouping of peaks at mass to charge ratio of about 1391 represents a single arginine pendant group functionalized to one C60 molecule, as ARG-C60 , and this is the primary reaction product. The grouping of peaks at mass to charge ratio of about 2036 represents a minor component of molecular fragments associated with two arginine groups functionalized to one C60 molecule, here designated as (ARG)2-C60 . The grouping of peaks at mass to charge ratio of about 2657 represents the trace amounts of molecular fragments associated with three arginine groups functionalized to one C60 molecule, here designated as (ARG)3-C60.

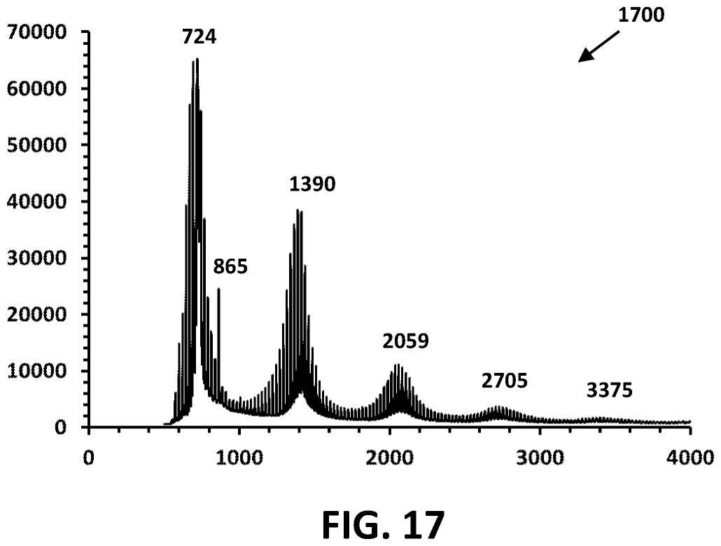

FIG. 17 presents experimental mass spectrograph data for the pivot adduct of arginine (ARG) derivatized C60 to adenosine triphosphate (ATP) derivatized C60 , here designated as the pivot ensemble of ARG-C60 -pivot-ATP-C60 . The largest molecular peak at about 724 mass to charge ratio represents the core molecule of C60 after all of the functional groups of each portion of the pivot have been ablated away with the exception of four endohedral bonded hydrogen atoms that face the interior of the cage structure and therefore are incapable of being removed by negative mode mass spectroscopy. This effect is also shown in FIG. 16 and is associated with the ARG-C60 portion of the pivot ensemble. The single peak at mass to charge ratio of about 865 represents a very characteristic molecular ion fragment associated with an arginine fragment that is still able to remain pendant to the remaining core C60 of part of the pivot ensemble. The grouping of peaks at mass to charge ratio of about 1390 represents the additive collective molecular fragments associated with one arginine group functionalized to one C60 core molecule as well as the molecular fragments of one adenosine triphosphate group functionalized to one C60 core molecule. The 1390 peak grouping is an adduct pivot and represents the primary composition of the ensemble of ARG-C60 -pivot-ATP-C60 as the primary collective reaction product having an about 69 percent final product reaction yield. The grouping of peaks at mass to charge ratio of about 2059 represents a minor number of molecular fragments associated with two ATP groups reacted to one C60 which is adducted as a pivot to a C60 derivatized with one arginine group, providing about 25 percent of the reaction product composition. The minor grouping of peaks at mass to charge ratio of about 2705 and about 3375 represents a trace number of higher molecular mass fragments summing to less than about 6 percent of this pivot ensemble composition, and can be associated with various greater permutations of one to three arginine and one to three ATP adducts to their respective C60 core molecules.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but defined in accordance with the foregoing claims appended hereto and their equivalents.

What is claimed is:

1. A formulation comprising:
a first fullerene covalently bonded to an adenosine phosphate.

2. The formulation of claim 1, wherein the first fullerene comprises a C60 fullerene or C70 fullerene.

3. The formulation of claim 1, wherein the adenosine phosphate comprises an adenosine triphosphate, adenosine diphosphate, adenosine monophosphate, or cyclic adenosine monophosphate.

4. The formulation of claim 3 wherein the first fullerene is additionally covalently bonded to a second adenosine phosphate.

5. The formulation of claim 4, further comprising:
a second fullerene van-der-Waals bonded to the first fullerene, wherein the second fullerene is also covalently bonded to a first amino acid.

6. The formulation of claim 5, wherein the first amino acid comprises arginine.

7. The formulation of claim 5, wherein the first amino acid comprises lysine.

8. The formulation of claim 7, wherein the second fullerene is also covalently bonded to a second amino acid comprising arginine.

9. A method of preparing an electrodynamic biaxially pivoting fullerene pivot molecules, comprising:
adding dry crystalline adenosine triphosphate powder to a dry crystalline fullerene powder in about a molar 1:1 ratio to form a first mixture thereof;
reacting the first mixture in a first shear grinding mill at greater than 1000 per second shearing rate at below 55° C. for about 15 minutes to produce fullerene-ATP;
dissolving the fullerene-ATP into water including at least 10% glycerol to make a fullerene-ATP dispersion;
adding a dry crystalline powder of a first amino acid functional group (R1 ) and a dry crystalline powder of a second amino acid functional group (R2 ) to dry crystalline fullerene powder in a molar ratio of about 1:1:1 to form a second mixture thereof;
reacting the second mixture in a second shear grinding mill under shear pressure below 55° C. for about 15 minutes to produce fullerene-R1-R2;
dissolving the fullerene-R1-R2 into water including at least 10% glycerol to make a fullerene-R1-R2 dispersion; and
combining the fullerene-ATP dispersion with the fullerene-R1-R2 dispersion in a mixer while applying ultrasonic radiation to the combined dispersions to produce an electrodynamic biaxial fullerene pivot.

10. The method of claim 9, wherein R1 comprises a first amine and R2 comprises a second amine.

11. The method of claim 10, wherein R1 comprises L-arginine and R2 comprises L-lysine.

12. The method of claim 9 further comprising combining a therapeutic constituent with the electrodynamic biaxial fullerene pivot.

13. The method of claim 12 wherein the therapeutic constituent comprises a drug, a Ga3+ ion, an antibody, or mRNA.

14. The method of claim 12 wherein combining the therapeutic constituent with the electrodynamic biaxial fullerene pivot includes adding the therapeutic constituent to the combination of the fullerene-ATP dispersion with the fullerene-R1-R2 dispersion in the mixer.

15. The method of claim 12 further comprising adding the electrodynamic biaxial fullerene pivot to a saline solution and adding the saline solution including the electrodynamic biaxial fullerene pivot to blood plasma.

16. A method of treatment, comprising:
administering to a patient a formulation of an electrodynamic biaxial fullerene pivot molecule including a retained therapeutic constituent; and
stimulating the electrodynamic biaxial fullerene pivot molecule to release the therapeutic constituent molecule to a treatment area of the patient by
applying RF electromagnetic radiation with a frequency of between about 5.0 GHz to about 11.5 GHz, or
applying an electric current, with a frequency of between about 5.0 GHz to about 11.5 GHz, or
applying infra-red electromagnetic radiation with a wavelength of about 800 nm to about 1000 nm.

17. The method of claim 16, wherein the frequency is between about 9.5 GHz to about 11.5 GHz.

18. The method of claim 16, wherein the applied RF radiation or electric current is provided at about 500 watts per liter.

19. The method of claim 16, wherein the formulation comprises blood plasma including the electrodynamic biaxial fullerene pivot retaining the therapeutic constituent.
